(19) United States
(12) Patent Application Publication
(10) Pub. No.: US 2009/0036748 A1
(43) Pub. Date: Feb. 5, 2009

(54) Testing Process

(76) Inventors: Eugene Pergament, Chicago, IL (US); Carol J. Wilson, West Yorkshire (GB); Philip Wyatt, Toronto (CA); Howard S. Cuckle, North Yorkshire (GB)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/850,314

(22) Filed: Sep. 5, 2007

(30) Foreign Application Priority Data
Aug. 2, 2007 (GB) 0715030.3

(51) Int. Cl. A61B 5/00; G06F 19/00; C12Q 1/68

(52) U.S. Cl. 600/300; 702/19; 435/6

(57) ABSTRACT

A method of determining an inclusive residual risk that a pregnancy is affected by at least one phenotypic disorder included in a disorder set is provided. The method includes calculating a prior risk for a disorder set, calculating posterior risks for the individual disorders or groups of disorders in the disorder set that can be screened for and/or diagnosed prenatally, and calculating an inclusive residual risk for the disorder set by combining the prior risks for disorders for which no tests have been performed and the individual posterior risks.

### RELATED APPLICATIONS

This application claims priority benefit of United Kingdom Application No. 0715030.3, filed Aug. 2, 2007

### FIELD OF THE INVENTION

The present invention relates to a method of performing prenatal testing to provide a risk of fetal anomalies. In particular, the present invention relates to a method of producing an inclusive residual risk that a pregnancy is affected by any severe congenital disorder, or by any subset of severe congenital disorder.

### BACKGROUND

Prenatal testing for significant chromosomal anomalies (aneuploidies, mosaicism and structural anomalies) is currently divided into screening and diagnosis stages. A screening test produces a risk that the pregnancy is affected by a specific disorder; this information is used to aid a decision (by medical practitioner and patient) whether to proceed to additional procedures such as invasive diagnostic testing, either chronic villus sampling (CVS) or amniocentesis. Generally the post-test estimated risk is compared to a fixed cutoff; an invasive test is recommended if the risk exceeds this cutoff. Prenatal testing is commonly performed for Down syndrome (Trisomy 21).

Down syndrome risk calculation is complex and requires dedicated software. It involves modification of the prior risk (usually estimated from maternal age and previous history) by a likelihood ratio estimated from measurements of a set of biochemical and/or ultrasound markers, and from other factors, such as maternal weight, to produce a posterior risk. Several variations on the basic method are in common use, including: choice of serum and/or ultrasound measurements, whether separate test results are combined in the calculation (including from different trimesters), various serum and ultrasound marker sets, differences in calculation methods and parameters used to estimate likelihood ratios, and whether posterior risk corresponds to gestational age at testing or pregnancy term.

The same basic method can be used to screen for other chromosomal anomalies, and in recent years, prenatal screening has been extended to include some of these. Currently, the risk for each disorder is calculated and reported separately.

Prenatal genetic screening is used to determine parental carrier status for some single-gene disorders such as cystic fibrosis. Currently, only pregnancies that are found to be at high risk (i.e. where both parents are carriers) usually proceed to diagnostic testing.

CVS and amniocentesis enable diagnosis of chromosomal anomalies by karyotyping or newer rapid techniques, and of single-gene disorders by genetic testing. They can also be used to supply material for expression arrays or Microarray-Based Comparative Genomic Hybridization testing (a-CGH), which are diagnostic methods that can detect more subtle molecular lesions, for example, DNA rearrangements and copy number variations, including clinically-significant microdeletions and microduplications. Where these techniques are used, the testing and reporting process is separate from the chromosomal testing process. Current and rapidly developing microarray technologies pose a serious problem for providers of prenatal testing, because of the enormous amount of information they produce, much of which is of uncertain clinical relevance.

For pregnancies assessed as low risk after screening and therefore not invasively tested, a detailed ultrasound examination is commonly carried out at around 18 to 20 weeks gestation. ‘Soft’ markers that are indicative but not diagnostic of chromosomal anomaly are sometimes found during the examination.

Fetal magnetic resonance imaging (MRI) is used (though not widely) as another independent screening or diagnostic test.

Although there is not yet a reliable method of obtaining fetal DNA non-invasively from maternal blood, many publications suggest that it will be feasible to introduce this into prenatal testing. This will enable much more extensive DNA testing than current normal practices.

Each of the above currently used screening methods may produce a risk for a specified disorder or group of disorders. However, the risks produced are usually reported separately. The number of different disorders that can be prenatally screened for, and the separate tests that are available, can be very confusing for patients, and reported test results are therefore not as useful as they could be in making pregnancy management decisions (including, for example, whether to proceed to further screening or diagnostic testing).

### SUMMARY OF THE INVENTION

It is an object of the present invention to address these and other problems associated with the prior art.

According to a first aspect of the present invention, there is provided a method of determining an inclusive residual risk of a pregnancy being affected by at least one phenotypic disorder included in a disorder set, the said method comprising the steps of:
a) selecting a disorder set and determining an appropriate scheduled prenatal test or tests for any specific disorders or groups of disorders within the disorder set that can be screened for and/or diagnosed prenatally;
b) calculating a prior risk for the disorder set;
c) calculating posterior risks for the specific disorders or groups of disorders in the disorder set that can be screened for and/or diagnosed prenatally, based on scheduled test results; and
d) calculating an inclusive residual risk for the disorder set by combining the prior risks for disorders within the disorder set for which no scheduled tests have been performed and the posterior risks for the disorders or groups of disorders within the disorder set for which scheduled tests have been performed.

Preferably, the inclusive residual risk is a measure of the posterior probability that the pregnancy is affected by one or more of the disorders in the selected disorder set. Preferably, the inclusive residual risk is presented as a quantitative risk value of 1 in some number, being the expected fraction of pregnancies which is affected by one or more of the disorders within the selected disorder set, according to the prior risk and test results.

### BRIEF DESCRIPTION OF THE DRAWINGS

FIG. 1 is a flow chart for the analysis carried out in Example 1.

FIG. 2 is a flow chart for the analysis carried out in Example 2.

### DETAILED DESCRIPTION

For the avoidance of doubt, the term ‘prior risk’, when used herein, refers to the risk of a pregnancy being affected by a disorder or group of disorders, based on population statistics and patient-specific clinical information and risk factors, for example maternal age and previous affected pregnancy, where risk is probability i.e. odds/(1+odds).

The term ‘posterior risk’, when used herein, refers to a modified prior risk for a disorder or group of disorders, based on the prior risk and the results of any screening and/or diagnostic tests which have been performed. Preferably, posterior risks may be 0 (no risk) or 1 (positive diagnosis) following diagnostic testing, or a value within these limits following non-diagnostic testing.

The term ‘inclusive residual risk’, when used herein, refers to an overall risk that the pregnancy is affected by at least one disorder in the disorder set of interest, being the combination of posterior risks for any disorder or group of disorders for which screening and/or diagnostic tests have been performed, and prior risks for any disorder or group of disorders in the set for which no prenatal screening or diagnostic tests have been performed. It may refer to such a risk estimated and reported following any step of a multi-stage testing process, or to the final risk estimated after the testing process has terminated.

The term ‘molecular lesion’, when used herein, includes DNA microdeletions, microduplications, rearrangements, point mutations, and any other clinically-significant DNA abnormalities detectable by microarray technology.

The term ‘prenatal test’, when used herein, refers to any screening or diagnostic test for fetal anomaly.

The term ‘group of disorders’, when used herein, refers to several fetal anomalies investigated together in the same prenatal test.

The disorder set may include all clinically significant congenital anomalies. Alternatively, the disorder set may comprise a subset of the clinically significant congenital anomalies. For example, the subset may be all serious chromosomal anomalies, or all serious genomic congenital disorders. The subset may comprise those anomalies considered to be relevant by the patient and/or medical practitioner. Preferably, the subset is determined by assessing the patient's clinical and family history.

Preferably, the scheduled screening tests include any screening tests which may be performed during pregnancy. Preferably, the scheduled screening tests include imaging screening, serum testing, parental carrier status screening for genetic disorders, and/or expression array or a-CGH DNA testing. Imaging screening may include ultrasound screening and/or magnetic resonance imaging screening. Any other suitable test may be performed as a scheduled test.

Preferably, the scheduled screening and diagnostic tests are appropriate to the disorder set. Depending on the disorder set, it may be beneficial to perform one or more scheduled tests. Preferably, multi-stage testing is performed. Multi-stage testing may include, but is not limited to, 1^{st} and 2^{nd} trimester serum screening tests, fetal imaging, diagnostic tests where indicated by High Risk screening results, and/or expression array or a-CGH testing.

The scheduled tests are preferably determined upon the selection of a disorder set.

Non-limiting examples of disorders which may be included in the disorder set are given in Table 1 below.

TABLE 1
Examples of disorders

| Type | Example |
|---|---|
| Chromosomal | Down syndrome |
| | Edwards syndrome |
| | Patau syndrome |
| | Turner syndrome |
| | Triploidy |
| Single-gene | Cystic fibrosis |
| | Sickle-cell disease |
| | Tay-Sachs disease |
| Multifactorial | Isolated neural tube defect |
| | Cardiovascular malformation |

Preferably, the results of the scheduled tests may be used to calculate and report risks of specific disorders or groups of disorders within the disorder set.

Preferably, the prior risk for the disorder set is calculated by combining the individual prior risks for each disorder within the set. Preferably, the prior risk is obtained from published reference figures. Preferably, the prior risk is corrected for maternal age at expected date of delivery for disorders within the disorder set for which published age-specific prevalence data is available. Preferably, the prior risk is modified for any other patient-specific details for which specific information is available, for example, any previous affected pregnancies, ethnicity and the like.

Preferably, the posterior risks are calculated by applying likelihood ratios to the prior risks, or by removing the prior risks, for any specific disorders or groups of disorders in the disorder set that can be screened or diagnosed prenatally.

Likelihood ratios are preferably obtained from the results of the scheduled tests. Preferably, an individual likelihood ratio is calculated for each disorder for which individual screening can be performed. Each likelihood ratio is calculated from a combination of marker results which are obtained from the tests. The marker result may be a measurement, or may be a simple Present or Absent. The same or different markers may be used in calculating likelihood ratios for different disorders.

Examples of suitable markers which may be used in determining likelihood ratios include, but are not limited to, those listed in table 2.

TABLE 2
Examples of marker sets

| Set | Marker | Abbreviation |
|---|---|---|
| 1st trimester | Pregnancy-Associated Plasma | PAPP-A |
| serum | Protein A | |
| | Free-beta Human Chronic | HCGb |
| | Gonadotropin | |
| | Human Chronic Gonadotropin | HCG |
| 1st trimester | Nuchal Translucency | NT |
| ultrasound | Nasal Bone | NB |
| 2^{nd} trimester | Alpha Fetoprotein | AFP |
| serum | Free-beta Human Chronic | HCGb |
| | Gonadotropin | |
| | Human Chronic Gonadotropin | HCG |
| | Unconjugated Estriol | UE3 |
| | Inhibin A | INHIB-A |
| 2^{nd} trimester | Nuchal Fold | NF |
| ultrasound | Echogenic Bowel | EB |
| | Echogenic Intracardiac | EICF |
| | Focus | |
| | Ventriculomegaly | VM |

The inclusive residual risk is preferably calculated by applying individual likelihood ratios in turn to the prior odds for each disorder or group of disorders that has been screened, to produce individual posterior odds, and by setting to 0 the posterior risk for any disorder or group of disorders that has been eliminated by diagnostic testing, and combining the prior risks for disorders within the disorder set for which no scheduled tests have been performed and the individual posterior risks.

For a disorder set wherein all of the disorders or groups of disorders are screened for prenatally, the inclusive residual risk is calculated by combining the posterior risks for each of the disorders or groups of disorders within the disorder set.

For a disorder set wherein none of the disorders are screened for prenatally, the inclusive residual risk is calculated by combining the prior risks for the disorders in the disorder set.

Preferably, the posterior risk for a specific disorder or group of disorders is modified by each step of a multi-stage testing process, and the inclusive residual risk at each stage accounts for the cumulative data received from all the steps undertaken so far. Therefore, the inclusive residual risk is also, preferably, modified by each step in a multi-stage testing process. The inclusive residual risk, and/or the posterior risks for individual disorders or groups that have been tested at a particular stage, may become the prior risks at the next stage. The same or different markers may be used at each testing stage.

The inclusive residual risk preferably provides information on whether to proceed to a further testing stage. The further testing stage may be further screening testing or may be diagnostic testing.

Preferably, when an inclusive residual risk is greater than a High Risk threshold, further diagnostic testing is recommended. For example, where the disorder set comprises all chromosomal anomalies, karyotyping may be recommended when an inclusive residual risk is greater than the threshold.

Alternatively, individual risks may be reported for individual disorders and/or groups of disorders, within the disorder set, to determine whether further testing is required, and if so, which further tests should be performed.

Preferably, any diagnostic testing performed may modify the inclusive residual risk. The modification may be a positive diagnosis or a removal of the risk associated with an individual disorder or group of disorders. Alternatively, the test result may enable risk modification by calculation of a likelihood ratio associated with an individual disorder or group of disorders.

Diagnostic testing may comprise karyotyping, expression array or a-CGH testing, other fetal DNA testing, fetal imaging or any other suitable diagnostic testing procedure.

Preferably, the inclusive residual risk calculation uses patient-specific, for example age- or ethnicity-related, prior risk where appropriate, and the population prior risk for other disorders or groups of disorders.

Preferably, the inclusive residual risk calculation provides a method of combining screening and diagnostic test results. Tests such as a-CGH and fetal imaging, which may be regarded as either screening or diagnostic tests, may be used to modify the inclusive residual risk at any stage. Preferably, therefore, the inclusive residual risk calculation removes the distinction between screening and diagnostic test procedures.

Preferably, the inclusive residual risk represents the risk of all adverse pregnancy outcomes, including, but not limited to, severe disorder and/or pregnancy loss.

Preferably, an inclusive residual risk is calculated and reported to the patient at each step of a multi-stage prenatal screening and/or diagnostic testing process.

Preferably, a final inclusive residual risk is calculated and reported to the patient after all the scheduled tests and any further tests have been performed, or after the testing process has terminated for any reason.

According to a second aspect of the present invention, there is provided a multi-stage prenatal testing process comprising the steps of:
a) selecting a disorder set and determining an appropriate schedule of screening and/or diagnostic tests for any specific disorders or groups of disorders within the disorder set that can be tested prenatally; the scheduled tests to include diagnostic testing if the inclusive residual risk or one or more individual risks for a disorder or group of disorders within the disorder set exceeds a high risk threshold following a scheduled screening test or tests;
b) performing scheduled prenatal tests;
c) calculating and reporting a modified inclusive residual risk of the pregnancy being affected by a disorder within a specified disorder set, and individual risks for disorders or groups of disorders in the disorder set, after each scheduled test in a stepwise risk refinement process; and
d) calculating a final inclusive residual risk, taking into account all prior and testing information, after the testing process has terminated.

Preferably, the scheduled screening tests comprise one or more of 1^{st} and/or 2^{nd} trimester serum screening, expression array or a-CGH DNA testing and/or fetal imaging screening, and further diagnostic tests where indicated by High Risk screening results.

The high risk threshold preferably depends on the disorders within the disorder set or the individual disorders or groups of disorders for which screening has been performed.

Preferably, the inclusive residual risk informs the decision whether the testing process should be terminated or continued. The testing process may be terminated because all possible tests have been performed, or at an earlier stage because the risk is deemed low enough by the patient that further tests are not requested, or for cost reasons.

The further diagnostic tests may include karyotyping, expression array or a-CGH testing, other fetal DNA testing, fetal imaging and/or any other suitable diagnostic testing procedure.

According to a third aspect of the present invention, there is provided a method of determining an inclusive residual risk of a pregnancy being affected by any severe congenital anomaly, or subset of severe congenital anomaly, including the finding or non-finding of molecular lesions from expression arrays or Microarray-Based Comparative Genomic Hybridization (a-CGH), the said method comprising the steps of:
a) calculating a prior risk for the pregnancy being affected by any severe congenital anomaly, or by any disorder in a disorder set of interest to the patient;
b) modifying the prior risk by calculating a likelihood ratio for each disorder or group of disorders in the set for which there are known associations with molecular lesion locations, and applying the likelihood ratios to the prior risk to give a posterior risk;
c) modifying the prior risk by estimating the proportion of prior risk for the disorder set that can be removed where a known proportion of a disorder or group of disorders is due to molecular lesions at specific locations, following normal findings at those locations, to give a posterior risk;
d) modifying the prior risk for disorders or groups of disorders, or for all significant congenital anomalies, where whole-genome array results are available and where molecular lesions of uncertain specific significance are found, to give a posterior risk; and
e) calculating the inclusive residual risk by combining the posterior risks for each disorder or group of disorders for which the finding or non-finding of molecular lesions provides information that can be used in risk modification, and the prior risks for each disorder or group of disorders for which it does not.

Preferably, the posterior risks are calculated by applying likelihood ratios to the prior risk for those disorders or groups of disorders for which there are known associations with molecular lesion locations. Preferably, the likelihood ratios are obtained from the frequency of the molecular lesion at each location in affected and unaffected fetuses.

Preferably, risk modification for disorders or groups of disorders where molecular lesions of uncertain significance are found, is by calculation of likelihood ratios, and these likelihood ratios are obtained from expression array or a-CGH data by a non-parametric lookup method. Preferably, the non-parametric lookup method uses the distributions of the total number and/or extent of such errors in affected and unaffected individuals.

Preferably, the modifications of the posterior risk may be performed in conjunction with analogous modifications from other screening or diagnostic test results. Examples of other suitable tests include, but are not limited to, serum, ultrasound, cytogenetic and/or other DNA tests.

According to a fourth aspect of the present invention, there is provided a final inclusive residual risk which provides a personalized risk of a pregnancy being affected by any disorder within a disorder set, taking into account information generated by any performed prenatal screening and/or diagnostic tests and prior risks for any disorders within the disorder set for which prenatal screening and/or diagnostic tests have or have not been performed.

The present invention also extends to a computer program product that when run is operable to perform a method of determining an inclusive residual risk of a pregnancy being affected by any phenotypic disorder included in a disorder set, the said method comprising the steps of:
a) calculating a prior risk for the disorder set;
b) calculating posterior risks for the specific disorders or groups of disorders in the disorder set that are screened for and/or diagnosed prenatally, based on scheduled test results;
c) calculating an inclusive residual risk for the disorder set at each step of a multi-stage testing process by combining the posterior risks for the disorders or groups of disorders that have been tested and the prior risks for the disorders that have not been tested; and
d) calculating a final inclusive residual risk for the disorder set after the testing process has terminated.

Preferably, the method further comprises the step of incorporating into the inclusive residual risk the finding or non-finding of molecular lesions from expression arrays or a-CGH.

All of the above features may be taken in any combination, and with any aspect of the invention.

The invention will now be described and illustrated by way of comparative examples, published methods and data, and non-limiting examples.

The following comparative examples, descriptions of prenatal screening and diagnostic tests, and published risk calculation methods and data, are non-limiting examples of existing and potential prenatal testing information that may be included in calculation of inclusive residual risk according to the present invention.

### COMPARATIVE EXAMPLE

### Risk Calculation for a Single Disorder (Down Syndrome—T21) Using Existing Methods

Prior Risk

A published equation is usually used, from regressed data for birth prevalence vs maternal age at estimated date of delivery (MAEDD); e.g.:

Age-related risk=0.000627+exp(−16.2395+0.286*(x−0.5)) where x is MAEDD in completed years. This equation and published prior risk figures can be found in Cuckle H S, Wald N J, Thompson S G, “Estimating a woman's risk of having a pregnancy associated with Down syndrome using her age and serum alpha-fetoprotein level.”, Br J Obs Gynaecol, 94:387-402, 1987.

Additive factors, which are MAEDD-independent, can be included in the prior risk calculation, usually corresponding to a previous pregnancy affected by a chromosomal anomaly. For example, a previous T21 pregnancy would increase the prior risk by 0.0042. See, for example, Cuckle H, Arbuzova S, “Multimarker Maternal Serum Screening for Chromosomal Abnormalities, in Milunsky A (ed): Genetic Disorders and the Fetus: Diagnosis, Prevention and Treatment (ed 5).”, Johns Hopkins University Press, USA, 2004, p 818.

Calculation of Likelihood Ratio (LR)

In order to standardize for gestational age variation, systematic differences between assays and laboratories (serum markers) or operators (ultrasound markers), continuous-valued measurements are normalized to multiples of the median (MoMs) or differences from the median (deltas), using a median equation by days gestation or ultrasound measurement; e.g.:

NT (mm)=10̂(−0.3599+0.0127*x−0.000058*x²) where x is Crown-Rump Length (CRL) in mm.

Such estimation correction parameters can be found in Nicolaides K H, Snijders R J M, Cuckle H S, “Correct estimation of parameters for ultrasound nuchal translucency screening.” Prenat Diagn, 18:511-523, 1998. By way of example, when NT=1.5 mm and CRL=70 mm, the median NT is 1.758, and therefore, the normalized NT (MoM) is 1.5/1.758=0.853 and the normalized NT (Delta) is 1.5−1.758 mm=−0.258 mm.

Multiplicative covariables can be used to adjust the marker median for a particular circumstance. The covariable is the expected MoM for that circumstance or condition. The covariable is usually a single marker-specific value corresponding to presence of the condition. For maternal weight, the covariable is calculated from an equation of expected MoM vs weight. Other covariables are obtained from published figures. Published covariables include maternal diabetes and maternal smoking, such as those published in Wald N J; Kennard A, Hackshaw A K, McGuire A, “Antenatal Screening for Down's Syndrome” Health Technology Assessment, 2:1, 1998, p 23 & 26.

LR is calculated from normalized marker values. For serum and other continuous-valued markers whose distribution in unaffected and affected pregnancies is Gaussian (or log Gaussian), the standard multivariate Gaussian model can be used, implemented with matrix mathematics of the type shown in Reynolds T M, Penney M D, “The mathematical basis of multivariate risk screening with special reference to screening for Down's syndrome associated pregnancy.” Ann Clin Biochem, 27:452-458, 1989.

Distribution parameters, i.e. means and standard deviations (SDs) for each marker and R-values for each marker pair in the set, are usually published values derived from meta-analysis. Examples of such published figures are given in Wald N J; Rodeck C; Hackshaw A K; Walters J; Chitty L, “First and second trimester antenatal screening for Down's syndrome: the results of the Serum, Urine and Ultrasound Screening Study (SURUSS)”, Health Technology Assessment, 7:11, 2003. Parameters may be specific to the number of completed weeks gestation (usually affected means) or to trimester.

Nuchal Translucency (NT) can be included in the multivariate Gaussian set. It is usually assumed to have a zero correlation with all serum markers. Alternatively a non-parametric method can be used to include NT, which uses a simple lookup table derived from a very large dataset to obtain LR from NT delta. The alternative approaches are discussed in Spencer K, Bindra R, Nix A B, Heath V, Nicolaides K H, “Delta-NT or NT MoM: which is the most appropriate method for calculating accurate patient-specific risks for trisomy 21 in the first trimester?”, Prenat Diag, 24(3):169-73, 2004.

For binary Normal/Abnormal imaging markers, LR can be estimated as follows:

LR corresponding to abnormal finding is:
(OddsAbnormalAff/(1+OddsAbnormalAff))/(OddsAbnormalUnaff/(1+OddsAbnormalUnaff)),

where OddsAbnormalAff is the odds of an abnormal finding in an affected fetus, and OddsAbnormalUnaff is the odds of an abnormal finding in an unaffected fetus.

LR corresponding to normal finding is:
(1−(OddsAbnormalAff/(1+OddsAbnormalAff)))/(1−(OddsAbnormalUnaff/(1+OddsAbnormalUnaff))).

Where LR depends in a complicated way on several imaging markers or other factors the method for calculation thereof can be extended by using an empirical relation for odds instead of a measured frequency. For example, as discussed in Cicero S, Rembouskos G, Vandecruys H, Hogg M, Nicolaides K H, “Likelihood ratio for trisomy 21 in fetuses with absent nasal bone at the 11-14-week scan.”, Ultrasound Obstet Gynecol, 23:218-223, 2004, for present (normal) and absent (abnormal) nasal bone (NB):
OddsAbnormalUnaff is exp(−0.367+1.582* (1 for Afro-Caribbean, 0 for other ethnic)−0.061*CRL+0.349*NT) where CRL is in mm and NT in deltas; and
OddsAbnormalAff is exp(2.275−0.032*CRL+0.207*NT)

By way of example, where NB=Abnormal, CRL=45 mm, NT delta=1.1 and Ethnic=Asian:
OddsAbnormalUnaff is exp(−0.367−0.061*45+0.349*1.1)=0.06534.
OddsAbnormalAff is exp(2.275−0.032*45+0.207*1.1)=2.894.

Therefore, LR is (2.894/(1+2.894))/(0.06534/(1+0.06534))=12.12.

Calculation of Posterior Risk

By the standard Bayesian method, Posterior odds=Prior odds*LR, so Posterior Risk=LR/(1/Prior Risk−1+LR). For example, when Prior Risk=0.005 and LR=2, the Posterior Risk is 2/(1/0.005−1+2)=0.00995.

Risk calculation can combine LRs derived by different methods, by multiplying the separate LRs, assuming that there is no marker correlation across the separate LRs.

Common refinements to the posterior risk calculation include, but are not limited to:
MoM Limits: The Gaussian distribution assumption is not valid for the tails of the distributions, so truncation limits may be applied. However, for values outside these limits, the limit is substituted for the actual value.
Twin Pregnancies: Both prior risk and likelihood ratio depend on zygosity, so this may be taken into account. In the case that the zygosity is not taken into account, the pregnancy is usually assumed to be dizygous. In dizygous pregnancies where the test includes a fetus-specific marker such as Nuchal Translucency, results are returned separately for each twin, and a fetus-specific prior risk is included. However, if only non-fetus-specific markers are included, the risk corresponds to at least one affected twin. In calculating Gaussian distributions for non-fetus-specific markers, twin-specific unaffected means are used, and the affected means are estimated from this data and the single-pregnancy affected means. Discussions of twin pregnancies are given in Cuckle H, Wilson C, “Twins—Risk per fetus or per pregnancy.”, Down's Screening News, 13:1:8, 2006, and Spencer K, “Screening for trisomy 21 in twin pregnancies in the first trimester using free beta-hCG and PAPP-A, combined with fetal nuchal translucency thickness.”, Prenat Diag, 20:91-95, 2000.
Combining results from tests at different gestations. As the marker values in LR calculation are normalized for gestation, the calculation method is unchanged when values from different gestations are combined. If the 1^{st} and 2^{nd} trimester tests are combined, gestation- or trimester-specific parameters must be used, including cross-trimester R-values.
Test and Term Risks. Risk at testing is calculated by dividing the term risk by a factor equal to the ratio of fetal survival rates in affected and unaffected pregnancies.

Other Disorders—Prior Risks and Existing Screening Methods

Calculation of Risk for Other Aneuploidies

Risk calculation for any other aneuploidy that has a known marker profile is performed as described for T21 above. Marker sets and gestational ranges may differ, and birth prevalence vs maternal age and distribution parameters for the aneuploidy are required. Examples of these equations and parameters are shown in Cuckle H, “Relative incidence of Down's, Edwards' and Patau's syndromes.”, Down's Screening News, 13:1:37, 2006 and Cuckle H, “Trisomies 18 & 13—Combined or separate risk.”, Down's Screening News, 13:2:15, 2006. Age-related combined prior risk of all serious aneuploidy is available from published meta-analysis such as that shown in Hook E B, Cross P K, Schreinemachers D M., “Chromosomal abnormality rates at amniocentesis and in live-born infants.”, JAMA, 249(15):2034-8, 1983. The overall population risk is about 1 in 500.

Screening for Single-Gene Disorders

Screening for single-gene disorders is not pregnancy-specific. The purpose of performing single-gene screening is to estimate a risk corresponding to the parents' carrier status. Taking Cystic Fibrosis (CF) as an example of a Mendelian autosomal recessive trait, birth prevalence in the UK is 1 in 2400 (Murray J, Cuckle H, Taylor G, Littlewood J, Hewison J., “Screening for cystic fibrosis.”, Health Technology Assessment, 3:8, 1999), implying a carrier frequency of 1 in 25. For a multiple-mutation assay with detection rate 0.8, 1 person in 125 tested is an undetected carrier. For a carrier couple, CF risk per pregnancy is 1 in 4, whereas for one carrier parent, where the other is found to have no mutations, the risk is 1 in 500, and where the other parent is not tested the risk is 1 in 100. If no mutation is found in either parent the risk is 1 in 62500.

For some single-gene disorders, presence of a mutation in the fetus may merely increase the risk of a disorder rather than being diagnostic of the disorder. For example, in Fragile X syndrome, all male full-mutation carriers are affected, whereas females have a risk of 0.5. Therefore, the presence or absence of mutation modifies the risk of severe mental retardation, as discussed in Murray J, Cuckle H, Taylor G, Hewison J, “Screening for fragile X syndrome.”, Health Technology Assessment, 1:4, 1997.

Risk For All Congenital Disorders

The World Health Organization (WHO) term classifies congenital anomalies as structural-morphological, functional and/or biochemical-molecular defects present at birth whether detected at that time or not. A typical figure (Hungary, 1908s) for the frequency of all major congenital anomalies in informative offspring (live born infants, stillborn fetuses and prenatally diagnosed and terminated affected fetuses is 27.01 per 1000 i.e. 1 in 37 as discussed in Czeizel A E, “Birth Defects Are Preventable.”, Int J Med Sci, 2:91-92, 2005. Disorders due to gene-environmental interaction and those of unknown origin, and so not prenatally diagnosable at present, such make up 60% of the total.

Examples of Diagnostic Testing Procedures

Cytogenetic and DNA Testing

The material used in the testing is obtained from Chronic villus sampling (CVS) which is performed during the latter stages of the 1^{st} trimester, or from amniocentesis which is performed during the 2^{nd} trimester. Fetal cells and cell-free DNA in maternal blood can be used to obtain material for cytogenetic and other DNA testing but, because of the small quantities, not reliably or effectively enough to replace invasive procedures. Simpson J L, Bisschoff F, “Intact Fetal Cells and Cell-free DNA in Maternal Blood, in Gogate S (ed): Preventive Genetics.” Jaypee Medical Publishers, India, 2005, p 314.

Cytogenetic traditional karyotyping tests are diagnostic. The posterior risk of any chromosomal anomaly arising from the karyotyping tests is, therefore, either 0 or 1. Rapid alternatives such as Quantitative Fluorescence Polymerase Chain Reaction testing (QF-PCR) currently cannot detect all cases of mosaicism and structural anomalies, so there is a post-test residual risk of chromosomal anomaly, as discussed in “Quantitative Fluorescent Polymerase Chain Reaction versus Cytogenetics: Risk-Related Indication and Clinical Implication of Nondetected Chromosomal Disorders.”, P. Kozlowski I. Grund G. Hickmann R. Stressig A. J. Knippel, Fetal Diagn Ther, 21:217-223, 2006.

Genetic Testing

Genetic testing for single gene disorders using material from amniocentesis or CVS is also considered diagnostic, though a residual risk generally remains following a negative result, as not all possible mutations are tested.

Examples of Available Prenatal Testing Information Not Currently Used in Risk Estimation.

a-CGH

Microarray-based comparative genomic hybridization (a-CGH) can detect molecular lesions, aneuploidies (not balanced alterations or mosaicism) and single-gene mutations. It is possible to screen for thousands of copy-number variations at once. In many cases the significance of the copy-number variations is unknown, but, for example, syndromes of mental retardation are rapidly being resolved into specific molecular lesions. Examples of such specific deletions or duplications are given in Aradhya S, Manning M A, Splendore A, Cherry A M, “Whole-genome array-CGH identifies novel contiguous gene deletions and duplications associated with developmental delay, mental retardation, and dysmorphic features.”, Am J Med Genet A, 143A:1431-1441, 2007. Providers of prenatal screening are unsure how to make use of the enormous amount information about the fetal genome potentially available from a-CGH, see for example Shuster E, “Microarray genetic screening: a prenatal roadblock for life?”, Lancet, 369:526-29, 2007.

Considering molecular lesions, either a normal or an abnormal a-CGH modifies the risk of congenital anomaly. For example, for a disorder associated with a specific molecular lesion location, if the association is not strong enough to be diagnostic, then positive and negative LRs could in principle be calculated from the frequency of the error in affected and unaffected fetuses, though this is not currently done.

Alternatively, if it is known what proportion of a disorder, group of disorders (for example, severe mental retardation or cardiac defects, or total congenital anomalies) is due to molecular lesions at specific locations, this part of the risk should be removed following a normal a-CGH; again, this is not currently done.

For the remainder of molecular lesions of uncertain significance, for example, from a whole-genome array, future studies into the distributions of total number of errors, and possibly the nature and extent of each lesion, in individuals with or without disorders, could provide data which would enable a non-parametric lookup method for obtaining LR for disorders or disorder groups from a-CGH data in the same way as T21 LR from NT delta.

Imaging

Both ultrasound “soft” markers and MRI markers can in principle be used to produce LRs for disorders or disorder groups, using the frequency of the marker in affected and unaffected fetuses. Some 2^{nd} trimester ultrasound LRs are published (Van den Hof M C, Wilson R D, Diagnostic Imaging Committee, Society of Obstetricians and Gynaecologists of Canada; Genetics Committee, Society of Obstetricians and Gynaecologists of Canada, “Fetal soft markers in obstetric ultrasound.”, J Obstet Gynaecol Can, 27(6):592-636, 2005), and it is likely that further imaging marker LRs will become available. This data, together with between-marker correlation data, will allow imaging marker results to be incorporated into inclusive residual risk calculation in the same way as serum markers.

Examples of Inclusive Residual Risk (IRR)

The following non-limiting examples show some applications and uses of the invention. In the examples, the prior risks, likelihood ratios (LRs) and posterior risks for individual disorders are calculated according to the above methods. Example calculation reference values are taken from the above published references. Some example calculation values are approximations based on inadequate published data; calculation values in the examples should be replaced as more precise data becomes available.

For simplicity of illustration only, it is assumed throughout the following examples that a pregnancy can be affected by at most one disorder.

The risks shown in bold are reported to the medical practitioner and patient.

### Example 1

Patient: Maternal age at estimated date of delivery (MAEDD)=35 years.

No previous affected pregnancy.

Disorder Set: All serious congenital.

Scheduled Tests: 1st trimester serum+ultrasound screening for T21, T18, T13; 2nd trimester serum screening for T21, T18, T13; 2nd trimester detailed ultrasound; karyotyping recommended if chromosomal anomaly risk above High Risk cutoff of 1 in 250.

1) Prior Risk Calculation

Typical published figure for all serious congenital disorders (not maternal age specific) is 1 in 37 i.e. odds 1:36. This is an average value across the MAEDD range, so is equivalent to the risk for an MAEDD in the middle range, i.e. approximately 30 years. The published figure includes some disorders for which age-specific data is available, and for these disorders, their contribution to the published overall value is replaced with age-specific values. The following method can be used to incorporate age-specific data for any disorder for which it is available. In this example age-specific prior odds for chromosomal anomalies are calculated and used in the overall prior odds.

Steps:

a) Calculate age-corrected prior odds using published age-specific values:

Prior odds for all chromosomal at MAEDD 30=1:500.

Prior odds for non-chromosomal serious congenital=Overall odds−Chromosomal odds=1:36−1:500=1:38.8.

Prior odds for all chromosomal at MAEDD 35=1:200.

Age-corrected combined prior odds=1:38.8+1:200=1:32.5, i.e. risk for MAEDD 35 is 1 in 34.

b) Calculate prior odds for all chromosomal except those disorders for which specific screening tests are scheduled:

Prior odds for T21+T18+T13 at MAEDD 35 are: 1:427 (T21), 1:3853 (T18), 1:11570 (T13). Combined odds=1:427+1:3853+1:11570=1:372, i.e. risk for T21+T18+T13 at MAEDD=35 is 1 in 373.

Prior odds for chromosomal excluding T21+T18+T13=1:200−1:372=1:433.

2) Scheduled Test Results

i) 1st Trimester Aneuploidy Screening (Serum+Ultrasound) at 12 Weeks Gestation

Free-beta human chronic gonadotropin (hCGb) 0.6 MoM;

Pregnancy-associated plasma protein A (PAPP-A) 0.5 MoM; NT=1.5 MoM.

ii) 2nd Trimester Aneuploidy Screening (Serum) at 15 Weeks Gestation

Alpha fetoprotein (AFP) 0.75 MoM; Human chronic gonadotropin (hCG) 1.2 MoM; Unconjugated estriol (uE3) 0.8 MoM.

iii) 2nd Trimester Detailed Ultrasound at 18 Weeks Gestation

Echogenic intracardiac focus (EICF) detected.

No other anomalies detected.

iv) Amniocentesis (karyotyping) at 19 Weeks Gestation

Normal karyotype.

3) Inclusive Residual Risk (IRR) Calculation Stages

i) IRR after 1^{st} trimester screening

a) Calculate posterior odds for disorders for which specific tests have been performed:

For serum markers, using published distribution parameters, LRs are: 0.3757 for T21, 0.3558 for T18 and 1.3207 for T13.

For ultrasound marker NT, using the non-parametric method, LRs are 1.309 for T21, 0.8375 for T18 and 0.3932 for T13. Combining serum and ultrasound LRs (assuming no serum-ultrasound marker correlation), LRs are: 0.3757*1.309=0.4917 for T21, 0.3558*0.8375=0.2980 for T18 and 1.3207*0.3932=0.5193 for T13.

Posterior odds for these aneuploidies are Prior odds*LR Therefore, the posterior odds are 1:427*0.4917=1:868 i.e. risk 1 in 869 for T21, 1:3853*0.2980=1:12930 i.e. risk 1 in 12930 for T18 and 1:11570*0.5193=1:22280 i.e. risk 1 in 22280 for T13.

Combined posterior odds for T21+T18+T13=1:868+1:12930+1:22280=1:785 i.e. risk 1 in 786.

b) Calculate combined posterior odds for all serious congenital disorders:

Combined posterior odds for all chromosomal=Prior odds for chromosomal excluding T21+T18+T13+Posterior odds for T21+T18+T13=1:433+1:785=1:279.

Combined posterior odds for all serious congenital=Prior odds excluding chromosomal+Combined posterior odds for chromosomal =1:38.8+1:279=1:34.1 i.e. IRR 1 in 35.

ii) IRR After 2^{nd} Trimester Serum Screening

a) Calculate posterior odds for disorders for which specific tests have been performed:

Combining all markers (serum and ultrasound) measured in both trimesters, using published distribution parameters, LRs are: 0.5910 for T21, 0.04802 for T18 and 0.3667 for T13.

Posterior odds (Prior odds * LR) are: 1:427*:0.5910=1:723 i.e. risk 1 in 724 for T21, 1:3853*0.04802=1:80240 i.e. risk 1 in 80240 for T18 and 1:11570*0.3667=1:31540 i.e. risk 1 in 31540 for T13.

Combined posterior odds for T21+T18+T13=1:723+1:80240+1:31540=1:700 i.e. risk 1 in 701.

b) Calculate combined posterior odds for all serious congenital disorders:
Combined posterior odds for all chromosomal=Prior odds for chromosomal excluding T21+T18+T13+Posterior odds for T21+T18+T13=1:433+1:700=1:268.

Combined posterior odds for all serious congenital=Prior odds excluding chromosomal+Combined posterior odds for chromosomal=1:38.8+1:268=1:33.9 i.e. IRR 1 in 35.

iii) IRR After 2^{nd} Trimester Ultrasound

a) Calculate posterior odds for all chromosomal:

Using the published data for the finding of EICF at ultrasound, LR for any aneuploidy is 2.

Applying this to 1^{st} trimester calculated odds for all chromosomal (assuming no correlation with any serum or other ultrasound marker tested), posterior odds for all chromosomal=1:268*2=1:134, i.e. risk 1 in 135.

b) Calculate combined posterior odds for all serious congenital disorders:

Combined posterior odds for all serious congenital=Prior odds excluding chromosomal+Posterior odds for all chromosomal=1:38.8+1:134=1:30.1, i.e. IRR 1 in 31.

iv) IRR After Amniocentesis (Karyotyping) Performed at 19 Weeks Gestation Due to Stage iii) IRR for Chromosomal Anomaly High Risk of 1 in 135.

Inclusive residual risk modified for karyotyping results: Prior odds for non-chromosomal serious congenital disorder=1:38.8 i.e. risk 1 in 40.

Normal karyotype removes chromosomal risk so final IRR for all serious congenital disorders is 1 in 40.

The procedure described in this example is set out in flow chart format in FIG. 1.

### Example 2

Patient: MAEDD=40 years

No previous affected pregnancy.

Disorder Set=All serious genomic.

Scheduled Tests: Cystic Fibrosis (CF) carrier screening; 2^{nd} trimester serum for T21; karyotyping recommended if T21 risk above High Risk cutoff of 1 in 250; genetic testing recommended if CF carrier screening indicates a High Risk category; a-CGH if invasive test performed.

1) Prior Risk Calculation

Typical published figure for all serious congenital disorders (not maternal age specific) is 1 in 37. Of this risk approximately 25% is due to currently known genomic causes, so risk of serious genomic disorder is 1 in 148 i.e. odds 1:147. As in example 1, assume the overall figure corresponds to MAEDD 30 and adjust for maternal age by substituting chromosomal component with age-related value.

Steps:

a) Calculate Age-Corrected Prior Odds:

Prior odds for all chromosomal at MAEDD 30=1:500.

Prior odds for non-chromosomal serious genomic=Overall genomic odds−Chromosomal odds=1:147−1:500=1:208.

Published prior odds for all chromosomal at MAEDD 40=1:67.

Age-corrected combined prior odds=1:208+1:67=1:50.7 i.e. risk 1 in 52.

b) Calculate prior odds for all chromosomal excluding those for which specific screening tests are scheduled:

Prior odds for T21 at MAEDD 40=1:128 i.e. risk 1 in 129.

Prior odds for chromosomal excluding T21 at MAEDD 40=Chromosomal odds−T21 odds=1:67−1:128=1:141.

2) Scheduled Test Results

i) CF Carrier Screening at 10 Weeks Gestation

Mother: carrier of delta-F508.

Father: not tested.

ii) 2nd Trimester Aneuploidy Screening (Serum) at 14 Weeks Gestation

Alpha-fetoprotein (AFP) 0.8 MoM

Human chronic gonadotropin (hCG) 1.1 MoM

Unconjugated estriol (uE3)=0.75 MoM.

iii) Amniocentesis (Karyotyping+CF Genetic Test) at 17 Weeks Gestation

Normal karyotype.

CF: no mutations detected.

iv) a-CGH at 19 Weeks

Microarray analysis using Bacterial Artificial Chromosome (BAC) clones for the subtelomeres, pericentromic regions and known genetic syndromes included showed no alterations for the loci tested.

3) Inclusive Residual Risk (IRR) Calculation

i) IRR After CF Carrier Screening

a) Calculate prior odds for non-chromosomal genomic excluding CF:

Published population prior odds for CF=1:2400.

Prior odds for non-chromosomal serious genomic excluding CF=Prior odds for non-chromosomal serious genomic−Population prior odds for CF=1:208−1:2400=1:228.

b) Correct combined posterior odds for CF carrier status:

From published carrier frequency 1 in 25, prior risk for CF, one carrier parent and the other not tested, is approximately 1 in 100 i.e. odds 1:99.

Odds for non-chromosomal serious genomic corrected for CF carrier status are 1:228+1:99=1:69.0.

Combined odds corrected for CF carrier status=Chromosomal prior odds+Non-chromosomal corrected odds=1:67+1:69=1:34.0 i.e. IRR 1 in 35.

ii) IRR after 2nd Trimester Screening

a) Calculate posterior odds for all chromosomal:

Using published distribution parameters, LR for T21 is 0.7082.

Posterior odds for T21=Prior odds*LR=1:128*0.7082=1:181 i.e. risk is 1 in 182.

b) Calculate combined posterior odds for all serious genomic disorders:

Posterior odds for all chromosomal=Prior odds for chromosomal excluding T21+Posterior odds for T21=1:141+1:181=1:79.1

Combined posterior odds for all serious genomic=Prior odds for non-chromosomal serious genomic+Posterior odds for all chromosomal=1:69+1:79.1=1:36.9 i.e. IRR 1 in 38.

iii) IRR After Karyotyping and Genetic Tests Performed Due to High 2 Trimester Risk for T21 of 1 in 182 and Mother's CF Carrier Status

Results show normal karyotype which removes chromosomal anomaly risk.

Normal result removes CF risk (multiple-mutation assay included parental mutation delta-F508).

Posterior odds for all serious genomic disorders=Prior odds for non-chromosomal serious genomic excluding CF=1:228 i.e. IRR 1 in 229.

iv) IRR After a-CGH

There is currently no accepted value for the proportion of all known serious genomic non-chromosomal anomaly that is detectable by a-CGH, because chips are expanding rapidly in capability and more clinically-significant genomic anomalies are being discovered. Typical advertised lists of loci and detection rates suggest that the value is of the order of 50%. A more precise chip-specific value should be used where known.

Steps:

IRR for non-chromosomal serious genomic after cytogenic tests=1 in 229.

As no alterations are shown in the a-CGH results, removing 50% of the IRR after cytogenic testing gives a posterior risk of 1/229*0.5=1 in 458, i.e. odds of 1:457.

Therefore, the final IRR for all serious genomic disorders is 1 in 458.

The genomic anomaly detection rate could be adjusted to take account of the exclusion of CF, but this is not necessary for such an approximate value.

The procedure followed in this example is set out in flow chart format in FIG. 2.

### Example 3

### Examples of General Applications of Inclusive Residual Risk

i) For a patient presenting a raised nuchal translucency after the first trimester ultrasound, and therefore having an increased risk of chromosomal anomalies and cardiac defects, the inclusive residual risk may represent the risk of these disorders. Depending on the risk at this stage, the inclusive residual risk could be modified, for example by performing a karyotyping diagnostic test to modify or remove the risk of chromosomal anomaly. The risk may be further modified by use of fetal imaging to provide a likelihood ratio or diagnosis of cardiac defects.

ii) For a patient who undergoes quantitative fluorescence polymerase chain reaction testing (QF-PCR), instead of full karyotyping, the final inclusive residual risk may represent the risk of any undetected serious chromosomal anomaly.

iii) Where there is a family history of severe mental retardation of unknown etiology, the inclusive residual risk may take into account the elimination of any known genomic causes, e.g. T21, Fragile X and molecular lesions, by diagnostic testing.

iv) Where one or both parents are of an ethnicity associated with higher risks of particular disorders, the inclusive residual risk may take account of such risks. For example, for an Ashkenazi Jewish couple, the final inclusive residual risk may represent the risk associated with undetected mutations after screening the couple for carrier status for genetic disorders of high frequency in Ashkenazi Jews.

v) Alternatively, the inclusive residual risk may represent the entire set of severe disorders which can, in principle, be diagnosed prenatally, for example chromosomal anomalies, single-gene disorders where the gene has been identified, and severe structural defects detectable by ultrasound including nural tube defects, cardiac, renal and other organ malformations.

The method of determining inclusive residual risk according to the present invention is advantageous because it extends current prenatal testing practice. The method dispenses with the separation of screening and diagnosis, giving a final result which is generally either a risk or a positive diagnosis.

The inclusive residual risk calculation integrates all test results into a single scheme, i.e. stepwise refinement of a single risk for the entire disorder set. This allows a meaningful, inclusive result to be returned at any stage of prenatal testing, which is currently not feasible.

The set of disorders included in the inclusive residual risk can contain all genomic anomalies of interest, for example chromosomal anomalies, single-gene disorders (using a population background prior risk or a carrier-specific risk), and molecular lesions such as microdeletions and microduplications. The inclusive residual risk is beneficial because the disorder set is not fixed by a particular clinic's practice, but can be specified by the patient and/or practitioner based on the patient's background and history. For example, the disorder set can include a group of disorders with known overall prior risk without the need for individual prior risks to be known, and disorders for which a test gives no likelihood ratio or diagnostic information.

Further, the inclusive residual risk calculation allows the a-CGH stage to be used either to provide a diagnosis or to estimate likelihood ratios or remove a fixed fraction of the risk for some disorders or groups of disorders, including those where the precise relation between genomic and phenotypic anomaly is not known. Also, detailed imaging, including MRI and the 2^{nd}-trimester ultrasound scan, can be used to estimate likelihood ratios from ‘soft’ markers and incorporate them into the inclusive residual risk. Also, the inclusive residual risk can incorporate patient-specific environmental risk factors, such as high alcohol intake.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method of estimating an inclusive residual risk, being a measure of the posterior probability that a pregnancy is affected by at least one phenotypic disorder included in a pre-selected disorder set, the said method comprising the steps of:
a) determining an appropriate scheduled prenatal test or tests for any specific disorders or groups of disorders within the disorder set that can be screened for and/or diagnosed prenatally;
b) calculating a prior risk for the disorder set;
c) calculating posterior risks for the specific disorders or groups of disorders in the disorder set that can be screened for and/or diagnosed prenatally, based on scheduled test results; and
d) calculating an inclusive residual risk for the disorder set by combining the prior risks for disorders within the disorder set for which no scheduled tests have been performed and the posterior risks for the disorders or groups of disorders within the disorder set for which scheduled tests have been performed.

2. The method according to claim 1, wherein the disorder set includes all clinically significant congenital anomalies.

3. The method according to claim 1, wherein the disorder set comprises a subset of the clinically significant congenital anomalies.

4. The method according to claim 1, wherein the scheduled screening tests include any screening tests which may be performed during pregnancy.

5. The method according to claim 1, wherein the scheduled tests may include 1^{st} and second trimester fetal imaging, 1^{st} and second trimester serum screening, parental carrier status screening for genetic disorders, diagnostic tests where indicated by High Risk screening results and/or expression array or a-CGH DNA testing.

6. The method according to claim 5, wherein multi-stage testing is performed, the stages including the tests according to claim 5, and wherein an inclusive residual risk is calculated and reported at each stage.

7. The method according to claim 1, wherein the results of the scheduled tests are used to calculate risks of specific disorders or groups of disorders within the disorder set.

8. The method according to claim 1, wherein the prior risk for the disorder set is calculated by combining the individual prior risks for each disorder or group of disorders within the set.

9. The method according to claim 1, wherein the prior risk is obtained from published reference figures.

10. The method according to claim 1, wherein the prior risk is modified for any patient-specific details for which specific information is available.

11. The method according to claim 1, wherein the posterior risks for disorders or groups of disorders are calculated by applying likelihood ratios to the prior risks, or by removing the prior risks, for any specific disorders or groups of disorders in the disorder set that can be screened or diagnosed prenatally.

12. The method according to claim 11, wherein the inclusive residual risk is calculated by applying individual likelihood ratios in turn to the prior odds for each disorder or group of disorders that has been screened, to produce individual posterior odds, and by setting to 0 the posterior risk for any disorder or group of disorders that has been eliminated by diagnostic testing, and combining the prior risks for disorders within the disorder set for which no scheduled tests have been performed and the individual posterior risks.

13. The method according to claim 1, wherein the posterior risk for a specific disorder or group of disorders is modified by each step of multi-stage testing.

14. The method according to claim 1, wherein the inclusive residual risk is modified by each step in the method, and accounts for the cumulative data received from all the steps undertaken so far.

15. The method according to claim 1, wherein the inclusive residual risk provides information on whether to proceed to a further testing stage.

16. The method according to claim 1, wherein separate risks are reported for individual disorders and/or groups of disorders within the disorder set, to determine whether further testing is required and, if so, which tests should be performed.

17. The method according to claim 1, wherein the inclusive residual risk calculation combines screening and diagnostic test results.

18. A multi-stage prenatal testing process comprising the steps of:
a) determining an appropriate schedule of screening and/or diagnostic tests for any specific disorders or groups of disorders within a preselected disorder set that can be tested prenatally; the scheduled tests to include diagnostic testing if the inclusive residual risk or one or more individual risks for a disorder or group of disorders within the disorder set exceeds a high risk threshold following a scheduled screening test or tests;
b) performing scheduled prenatal tests;
c) calculating a modified inclusive residual risk of the pregnancy being affected by a disorder within a specified disorder set, and individual risks for disorders or groups of disorders in the disorder set, after each scheduled test in a stepwise risk refinement process; and
d) calculating a final inclusive residual risk, taking into account all prior and testing information, after the testing process has terminated.

19. The process according to claim 18, wherein the scheduled screening tests comprise one or more of 1^{st} and/or 2^{nd} trimester serum screening, expression array or a-CGH DNA testing and/or fetal imaging screening, and further diagnostic tests where indicated by High Risk screening results.

20. A method of determining an inclusive residual risk of a pregnancy being affected by any severe congenital anomaly, or subset of severe congenital anomaly, including a finding or non-finding of molecular lesions from expression arrays or Microarray-Based Comparative Genomic Hybridization (a-CGH), the said method comprising the steps of:
a) calculating a prior risk for the pregnancy being affected by any severe congenital anomaly, or by any disorder in a disorder set of interest to the patient;
b) modifying the prior risk by calculating a likelihood ratio for each disorder or group of disorders in the set for which there are known associations with molecular lesion locations, and applying the likelihood ratios to the prior risk to give a posterior risk;
c) modifying the prior risk by estimating the proportion of prior risk for the disorder set that can be removed where a known proportion of a disorder or group of disorders is due to molecular lesions at specific locations, following normal findings at those locations, to give a posterior risk;
d) modifying the prior risk for disorders or groups of disorders, or for all significant congenital anomalies, where whole-genome array results are available and where molecular lesions of uncertain specific significance are found, to give a posterior risk; and
e) calculating the inclusive residual risk by combining the posterior risks for each disorder or group of disorders for which the finding or non-finding of molecular lesions provides information that can be used in risk modification, and the prior risks for each disorder or group of disorders for which it does not.

21. The method according to claim 20, wherein the posterior risks are calculated by applying likelihood ratios to the prior risk for those disorders or groups of disorders for which there are known associations with molecular lesion locations.

22. The method according to claim 21, wherein the likelihood ratios are obtained from the frequency of the molecular lesion at each location in affected and unaffected fetuses.

23. The method according to claim 20, wherein risk modification for disorders or groups of disorders where molecular lesions of uncertain significance are found is by calculation of likelihood ratios, and these likelihood ratios are obtained from a-CGH data by a non-parametric lookup method.

24. The method according to claim 20, wherein the modifications of the posterior risk are performed in conjunction with analogous modifications from other screening or diagnostic test results.

25. A final inclusive residual risk which provides a personalized risk of a pregnancy being affected by any disorder within a disorder set, taking into account information generated by any performed prenatal screening and/or diagnostic tests and prior risks for any disorders within the disorder set for which prenatal screening and/or diagnostic tests have or have not been performed.

26. A computer program product that when run is operable to perform a method of determining an inclusive residual risk of a pregnancy being affected by any phenotypic disorder included in a disorder set, the said method comprising the steps of:
a) calculating a prior risk for the disorder set;
b) calculating posterior risks for the specific disorders or groups of disorders in the disorder set that are screened for and/or diagnosed prenatally, based on scheduled test results;
c) calculating an inclusive residual risk for the disorder set at each step of a multi-stage testing process by combining the posterior risks for the disorders or groups of disorders that have been tested and the prior risks for the disorders that have not been tested; and
d) calculating a final inclusive residual risk for the disorder set after the testing process has terminated.

27. The method according to claim 26, further comprising the step of incorporating into the inclusive residual risk the finding or non-finding of molecular lesions from expression arrays or a-CGH.
